(19) United States
(12) Patent Application Publication
(10) Pub. No.: US 2004/0127715 A1
(43) Pub. Date: Jul. 1, 2004

(54) Adamantane compounds and insulating film forming coating solutions

(75) Inventors: Nobutaka Kunimi, Tsukuba-shi (JP); Yuji Yoshida, Edogawa-ku (JP)

Correspondence Address: SUGHRUE MION, PLLC, 2100 PENNSYLVANIA AVENUE, N.W., SUITE 800, WASHINGTON, DC, 20037, US

(73) Assignee: SUMITOMO CHEMICAL COMPANY, LIMITED

(21) Appl. No.: 10/624,547

(22) Filed: Jul. 23, 2003

(30) Foreign Application Priority Data
Jul. 25, 2002 (JP) 2002-216356
Dec. 25, 2002 (JP) 2002-374026
Dec. 25, 2002 (JP) 2002-374027
Dec. 25, 2002 (JP) 2002-374028

(51) Int. Cl. C07D 211/68

(52) U.S. Cl. 546/285

(57) ABSTRACT

The object of the present invention is to provide a novel compound which can prepare an insulating film having a low dielectric constant and excellent in the heat resistance. This object is achieved by an adamantane compound represented by the formula (1).

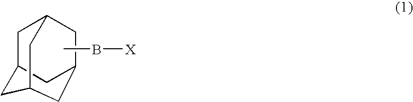

(wherein B represents a direct bond, an alkylene group having a carbon number of 1 to 6, an alkenylene group having a carbon number of 2 to 6, or an alkynylene group having a carbon number of 2 to 6, and X represents a monovalent organic group represented by the formula (2).

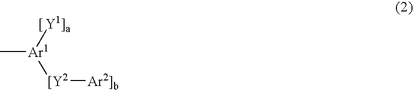

### FIELD OF THE INVENTION

The present invention relates to adamantan compounds, adamantane resins and insulating film forming coating solutions containing the adamantane compounds and the adamantane resins.

### BACKGROUND OF THE INVENTION

Recently, with making wiring minute in a semiconductor device, a transmitting rate of an electronic signal is delayed, which is so-called wiring retardation. In order to improve wiring retardation, there are strategies such as improvement in the performance of a wiring itself, reduction in interference between wirings and the like. As a method of reducing interference between wirings, there is improvement in the insulating performance of an insulating film. For improving the insulating performance, it is desired to develop an insulating film having a lower specific dielectric constant.

Since an organic material has a low specific dielectric constant, it is paid an attention as a material for an insulating film. Benzocyclobutene resins are known, but the resins have specific dielectric constant of around 2.7, and the insulating performance thereof is not sufficient. Therefore, it is desired to develop a novel compound which can prepare an insulating film having a low dielectric constant.

In addition, since thermal processing steps are included upon preparation of a semiconductor chip, thermal stability is required for an insulating film. When thermal stability of an insulating film is low, the insulating film cannot withstand thermal processing steps, and reliability of the semiconductor chip is deteriorated.

As an insulating film forming coating solution, there is a coating solution containing a polyimide resin (JP05-121396A) However, when it is prepared into an insulating film, a specific dielectric constant is not sufficient yet.

Therefore, it is desired to develop materials for forming an insulating film which can provide an insulating film having a low specific dielectric constant and high thermal stability.

### SUMMARY OF THE INVENTION

One of the objects of the present invention is to provide novel compounds which can provide insulating films having a low dielectric constant.

Another object of the present invention is to provide resins which can be used for insulating films having a low specific dielectric constant and high thermal stability.

And other object of the present invention is to provide coating solutions which can produce insulating films having a low specific dielectric constant and high thermal stability.

In order to find out the above mentioned novel compounds, the present inventors intensively studied and, as a result, found that adamantane compounds having a particular structure provides insulating films having a low dielectric constant, which resulted in completion of the present invention.

That is, the present invention provides adamantane compounds represented by the formula (1), and the adamantane resins comprising the adamantane compounds represented by the formula (1).

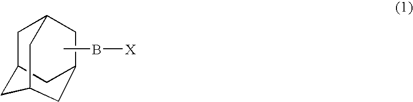

(wherein B represents a direct bond, an alkylene group having a carbon number of 1 to 6, an alkenylene group having a carbon number of 2 to 6, or an alkynylene group having a carbon number of 2 to 6, and X represents an organic group represented by the formula (2).

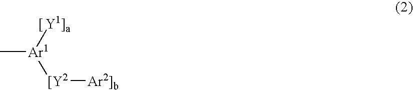

(wherein Ar¹ represents an optionally substituted three or more valent aromatic ring, Ar² represents an optionally substituted aryl group or an organic group represented by the formula (3), Y¹ represents an alkenyl group having a carbon number of 2 to 6or an alkynyl group having a carbon number of 2 to 6, Y² represents an alkenylene group having a carbon number of 2 to 6 or an alkynylene group having a carbon number of 2 to 6, and a and b represent an integer of 0 to 5, respectively, a+b is from 2 to 5.)

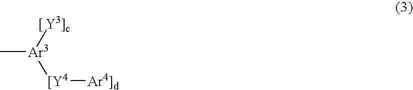

(wherein Ar³ represents an optionally substituted two or more valent aromatic ring, Ar⁴ represents an optionally substituted aryl group or an organic group represented by the formula (4), Y³ represents an alkenyl group having a carbon number of 2 to 6or an alkynyl group having a carbon number of 2 to 6, Y⁴ represents an alkenylene group having a carbon number of 2 to 6 or an alkynylene group having a carbon number of 2 to 6, and c and d represent an integer of 0 to 5, respectively, c+d is from 1 to 5.)

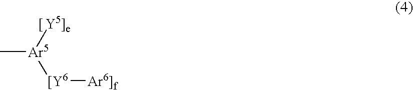

(wherein Ar⁵ represents an optionally substituted two or more valent aromatic ring, Ar⁶represents an optionally substituted aryl group, Y⁵ represents an alkenyl group having a carbon number of 2 to 6 or an alkynyl group having a carbon number of 2 to 6, Y⁶ represents an alkenylene group having a carbon number of 2 to 6 or an alkynylene group having a carbon number of 2 to 6, and e and f represent an integer of 0 to 5, respectively, e+f is from 1 to 5.)

Further, in order to find out the above mentioned resin, the present inventors intensively studied and, as a result, found that, when an adamantane resin obtained by polymerizing an above mentioned adamantane compound is used, an insulating film which has a low specific dielectric constant and is excellent in the heat resistance can be obtained, which resulted in completion of the present invention.

That is, the present invention provides adamantane resins, which are obtained by polymerizing a mixture comprising at least one kind adamantane compound represented by the formula (1), at least one kind adamantane compound represented by the formula (9) and/or at least one kind adamantane compound represented by the formula (10).

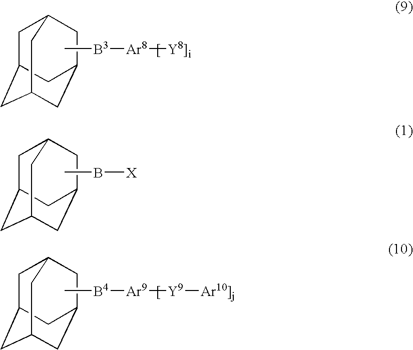

(wherein B and X are as defined above, B³ and B⁴ represent independently a direct bond, an alkylene group having a carbon number of 1 to 6, an alkenylene group having a carbon number of 2 to 6 or an alkynylene group having a carbon number of 2 to 6, Ar⁸ and Ar⁹ represent independently an optionally substituted three or more valent aromatic ring, Ar¹⁰ represents an optionally substituted aryl group, Y⁸ represents an alkenyl group having a carbon number of 2 to 6 or an alkynylene group having a carbon number of 2 to 6, Y⁹ represents an alkenylene group having a carbon number of 2 to 6 or an alkynylene group having a carbon number of 2 to 6, and i and j represent an integer of 2 to 5.)

Additionally, in order to find out the above mentioned coating solution, the present inventors intensively studied and, as a result, found that a coating solution for insulating film comprising a resin obtained by polymerizing an above mentioned novel adamantane compound represented by the formula (1), and at least one adamantane compound selected from the group consisting of adamantane compounds represented by the formula (1) and adamantane compound represented by the following formula (6).

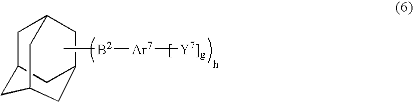

(wherein B² represent a direct bond, an alkylene group having a carbon number of 1 to 6, an alkenylene group having a carbon number of 2 to 6 or an alkynylene group having a carbon number of 2 to 6, Ar⁷ represents an optionally substituted two or more valent aromatic ring, Y⁷ represents independently an alkenyl group having a carbon number of 2 to 6 or an alkynyl group having a carbon number of 2 to 6, g represents an integer or 1 to 5, and h represents an integer of 2 to 4).

### DETAILED DESCRIPTION OF THE PRESENT INVENTION

A novel adamantane compound of the present invention is represented by the above formula (1).

In the formula (1), B represents a direct bond, an alkylene group having a carbon number of 1 to 6, an alkenylene group having a carbon number of 2 to 6, or an alkynylene group having a carbon number of 2 to 6. B is preferably a direct bond.

Examples of an alkylene group having a carbon number of 1 to 6 include a methylene group, an ethylene group, a propylene group, a butylene group and the like.

Examples of an alkenylene group having a carbon number of 2 to 6 include a vinylene group, a propenylene group, a butenylene group and the like.

Examples of an alkynylene group having a carbon number of 2 to 6 include an ethynylene group, a propynylene group, a butynylene group, a butadienylene group and the like.

An alkylene group, an alkenylene group, and an alkynylene group may be straight or branched and, when those groups have a double bond or a triple bond, positions of those bonds are not particularly limited.

X represents an organic group represented by the formula (2).

In the formula (2), Ar¹ represents an optionally substituted three or more valent aromatic ring. Examples of a three or more valent aromatic ring include those shown below.

The following formulas show a bond of a naphthalene ring and an anthracene ring may take an arbitrary position.

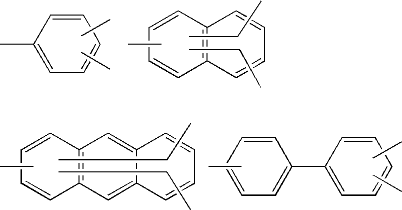

The above aromatic ring may be substituted with at least one kind substituent selected from the group of an alkyl group such as a methyl group, an ethyl group and the like; an alkoxy group such as a methoxy group, an ethoxy group and the like; a halogeno group such as a fluoro group, a chloro group, a bromo group and the like; an acyl group such as an acetyl group, a propionyl group and the like; an alkoxycarbonyl group such as a methoxycarbonyl group, an ethoxycarbonyl group and the like; a hydroxy group; a phenoxy group; a nitro group; a cyano group; a carboxy group; an amino group.

Y¹ represents an alkenyl group having a carbon number of 2 to 6 or an alkynyl group having a carbon umber of 2 to 6. Examples of an alkenyl group having a carbon number of 2 to 6 include a vinyl group, an allyl group, an isopropenyl group, a butenyl group, a butadienyl group, a hexenyl group and the like.

Examples of an alkynyl group having a carbon number of 2 to 6 include an ethynyl group, a propynyl group, an isopropynyl group, a butynyl group, a hexynyl group and the like. An alkenyl group and an alkynyl group may be straight or branched, and positions of a double bond and a triple bond are not particularly limited.

Y² represents an alkenylene group having a carbon number of 2 to 6 or an alkynylene group having a carbon umber of 2 to 6. Examples of an alkenylene group having a carbon number of 2 to 6 and an alkynylene group having a carbon number of 2 to 6 include those described above.

Ar² represents an optionally substituted aryl group or an organic group represented by the formula (3).

Examples of an aryl group include a phenyl group, a naphthyl group, an anthracenyl group and a biphenyl group, these groups may be substituted with at least one kind substituent selected from the group of an alkyl group such as a methyl group, an ethyl group and the like; an alkoxy group such as a methoxy group, an ethoxy group and the like; a halogeno group such as a fluoro group, a chloro group, a bromo group and the like; an acyl group such as an acetyl group, a propionyl group and the like; an alkoxycarbonyl group such as a methoxycarbonyl group, an ethoxycarbonyl group and the like; a hydroxy group; a phenoxy group; a nitro group; a cyano group; a carboxy group; an amino group.

Examples thereof include a phenyl group, a methylphenyl group, dimethylphenyl group, an ethylphenyl group, a diethylphenyl group, a trimethylphenyl group, a tetramethylphenyl group, a pentamethylphenyl group, a hydroxyphenyl group, a methoxyphenyl group, an ethoxyphenyl group, a phenoxyphenyl group, a fluorophenyl group, a chlorophenyl group, a bromophenyl group, an iodophenyl group, a nitrophenyl group, a cyanophenyl group, a carboxyphenyl group, a methyloxycarbonylphenyl group, an aminophenyl group, a naphthyl group, a methylnaphthyl group, a dimethylnaphthyl group, an ethylnaphthyl group, a diethylnaphthyl group, a trimethylnaphthyl group, a tetramethylnaphthyl group, a pentamethylnaphthyl group, a hydroxynaphthyl group, a methoxynaphthyl group, an ethoxynaphthyl group, a phenoxynaphthyl group, a fluoronaphthyl group, a chloronaphthyl group, a bromonaphthyl group, an iodonaphthyl group, a nitronaphthyl group, a cyanonaphthyl group, a carboxynaphthyl group, a methyloxycarbonylnaphthyl group, an aminonaphthyl group, a biphenyl group, an anthracenyl group and the like.

Each of a and b represents an integer of 0 to 5, and a+b is from 2 to 5., a+b is preferably 2 or 3.

In the formula (3), Y³represents an alkenyl group having a carbon number of 2 to 6 or an alkynyl group having a carbon number of 2 to 6. Examples of an alkenyl group having a carbon number of 2 to 6 and an alkynyl group having a carbon number of 2 to 6 include those as described above.

Y⁴ represents an alkenylene group having a carbon number of 2 to 6 or an alkynylene group having a carbon number of 2 to 6. Examples of an alkenylene group having a carbon number of 2 to 6 and an alkynylene group having a carbon number of 2 to 6 include those as described above.

Ar³represent an optionally substituted two or more valent aromatic ring.

Examples of a two or more valent aromatic ring include those described below.

The following formulas show a bond in a naphthalene ring and an anthracene ring represents may take an arbitrary position.

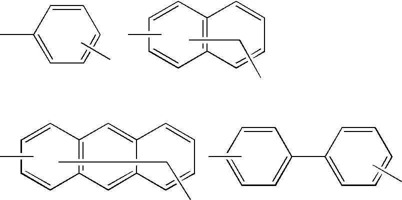

The above aromatic ring may be substituted with at least one kind substituent selected from the group of an alkyl group such as a methyl group, an ethyl group and the like; an alkoxy group such as a methoxy group, an ethoxy group and the like; a halogeno group such as a fluoro group, a chloro group, a bromo group and the like; an acyl group such as an acetyl group, a propionyl group and the like; an alkoxycarbonyl group such as a methoxycarbonyl group, an ethoxycarbonyl group and the like; a hydroxy group; a phenoxy group; a nitro group; a cyano group; a carboxy group; an amino group.

Ar⁴ represents an optionally substituted aryl group or an organic group represented by formula (4). Examples of an optionally substituted aryl group include those as described above.

Each of c and d represents an integer of 0 to 5, and c+d is from 1 to 5. c+d is preferably 1 or 2.

In the formula (4), Y⁵ represents an alkenyl group having a carbon number of 2 to 6 or an alkynyl group having a carbon number of 2 to 6. Examples of an alkenyl group having a carbon number of 2 to 6 and an alkynyl group having a carbon number of 2 to 6 include those as described above.

Y⁶ represents an alkenylene group having a carbon number of 2 to 6 or an alkynylene group having a carbon number of 2 to 6. Examples of an alkenylene group having a carbon number of 2 to 6 and an alkynylene group having a carbon number of 2 to 6 include those as described above.

Ar⁵ represents an optionally substituted two or more valent aromatic ring. Examples of a two or more valent aromatic ring include those as described above.

Ar⁶ represents an optionally substituted aryl group.

Examples of an optionally substituted aryl group include those as described above.

Each of e and f represents an integer of 0 to 5, and e+f is from 1 to 5. e+f is preferably 1 or 2.

X preferably contains at least one carbon-carbon triple bond since the reactivity at polymerization is enhanced, and it is more preferable that Y¹ is an alkynyl group having a carbon number of 2 to 6, and Y² is an alkynylene group having a carbon number of 2 to 6.

Specifically, it is more preferable that X is an organic group selected from the following group.

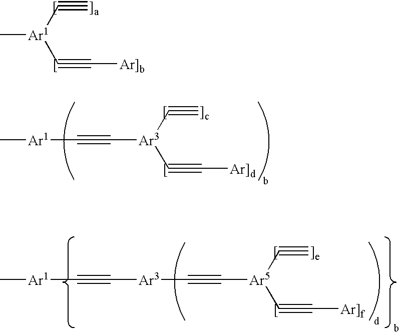

(wherein Ar¹, Ar³, Ar⁵, a, b, c, d, e and f are as defined above, and Ar represents an optionally substituted aryl group.)

When X is an organic group selected from the above groups, from the viewpoint that alkynylene groups are reacted with each other to form an aromatic ring, or form a polyvinylene chain or a polyacetylene chain, the mechanical strength of the resulting insulating film is enhanced.

In addition, it is more preferable that X is an organic group selected from the following group.

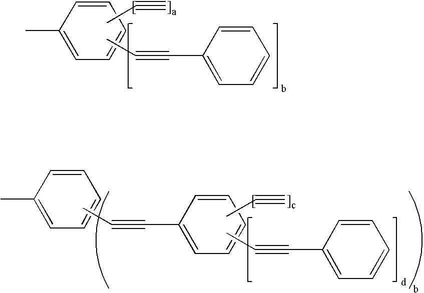

(wherein a, b, c and d are as defined above.)

When X is an organic group selected from the above group, an insulating film having the lower polarizing property and a lower specific dielectric constant may be obtained.

Further, from the viewpoint that acetylene, ethynylbenzene, diethynylbenene and the like are easily available as a raw material for adamantane compound, it is particularly preferable that X is an organic group selected from the following groups.

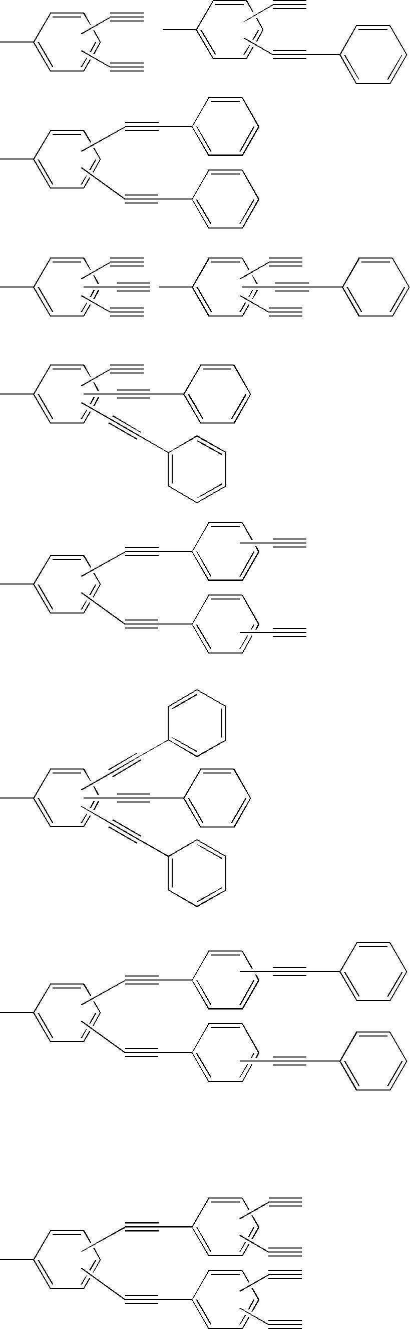

From the viewpoint that starting materials are industrially easily available and are easily prepared, it is particularly preferable that the compound represented by the formula (1) is a compound represented by the formula (5).

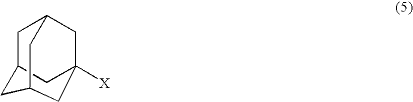

(wherein X is as defined above)

Examples of the compound represented by the formula (5) include diethynylphenyladamantane,

ethynyl-phenylethynylphenyladamantane,

di(phenylethynyl)phenyladamantane,

triethynylphenyladamantane,

di-ethynyl-phenylethynylphenyladamantane,

ethynyl-di(phenylethynyl)phenyladamantane,

tri(phenylethynyl)phenyladamantane,

bis(ethynylphenylethynyl)phenyladamantane,

bis(diethynylphenylethynyl)phenyladamantane,

bis (phenylethynylphenylethynyl) phenyladamantane and the like.

Among compounds represented by the formula (1), compounds having a substituent —B—X can be prepared, for example, by reacting 2-halogenoadamantane, with a compound which is prepared by activating a hydrogen atom of B (or a hydrogen atom of X when B is a direct bond) with lithium or the like. 2-halogenoadamantane as a raw material can be prepared, for example, by oxidizing a methylene group of adamantane with a strong acid such as sulfuric acid, nitric acid, fuming sulfuric acid and the like to convert into a carbonyl group, hydrogenating the group to a hydroxy group, and halogenating this hydroxy group with chlorine, bromine, iodine or the like.

In addition, among compounds represented by the formulas (1), compounds having a substituent —B—X at a bridging methine group of adamantane can be prepared, for example, by coupling 1-halogenoadamantane, with H—B—X. Upon a coupling reaction relating such the adamantane, Lewis acids such as aluminium chloride, tin chloride, antimony chloride, titanium chloride, aluminium bromide, tin bromide, antimony bromide, titanium bromide and the like may be used as a catalyst, ort-butyl chloride, t-butyl bromide, t-butyl iodide or the like may be present in addition to the catalyst.

1-halogenoadamantane as a raw material can be prepared, for example, by halogenating a bridging methine group of adamantane with chlorine, bromine, iodine or the like by the conventional method.

A coating solution can be obtained by dissolving the compound represented by the formula (1), a resin obtained by polymerizing the compound, or a mixture of them in an organic solvent.

As a method for polymerizing the compound represented by the formula (1), the known polymerization methods can be applied. Examples thereof include radical polymerization using a radical initiator such as benzoyl peroxide, t-butyl peroxide, azobisisobutyronitrile and the like, cation polymerization using a catalyst such as sulfuric acid, phosphoric acid, triethylaluminium and the like, anion polymerization using a catalyst such as lithiumnaphthalene and the like, photoradical polymerization using light irradiation and the like, thermal polymerization by heating, catalytic polymerization using a metal or a metal complex such as palladium, nickel, iron, cobalt and the like, and the like.

Polymerization usually proceeds by reacting Xs in the compound represented by formula (1). Examples of the resulting resin include poly(diethynylphenyladamantane),

poly(ethynyl-phenylethynylphenyladamantane),

poly(di(phenylethynyl)phenyladamantane),

poly(triethynylphenyladamantane),

poly(diethynyl-phenylethynylphenyladamantane),

poly(ethynyl-di(phenylethynyl)phenyladamantane),

poly(tri(phenylethynyl)phenyladamantane),

poly(bis(ethynylphenylethynyl)phenyladamantane),

poly(bis(diethynylphenylethynyl)phenyladamantane),

poly(bis(phenylethynylphenylethynyl)phenyladamantane) and the like.

Examples of an organic solvent used for obtaining a coating solution are not particularly limited, but alcohol solvents such as methanol, ethanol, isopropanol, 1-butanol, 2-ethoxymethanol, 3-methoxypropanol and the like; ketone solvents such as acetyl acetone, methyl ethyl ketone, methyl isobutyl ketone, 2-pentanone, 3-pentanone, 2-heptanone, 3-heptanone and the like; ester solvents such as ethyl acetate, propyl acetate, butyl acetate, isobutyl acetate, pentyl acetate, ethyl propionate, propyl propionate, butyl propionate, isobutyl propionate, propylene glycolmonomethyl ether acetate, methyl lactate, ethyl lactate, γ-butyrolactone and the like; ether solvents such as diisopropylether, dibutyl ether, ethyl propyl ether, anisole, phenetole, veratrole and the like; aromatic hydrocarbon solvents such as mesitylene, ethyl benzene, diethyl benzene, propyl benzene and the like are preferable as a solvent because they are industrially available. These may be used a lone or by mixing two or more.

Further, additives such as a radical generator, a nonionic surfactant, a non-ionic surfactant containing fluorine, a silane coupling agent and the like may be added to the coating solution in a range that the reactivity, the coating property and the like of compounds represented by the formulas (1) are not deteriorated.

Example of a radical generator include t-butyl peroxide, pentyl peroxide, hexyl peroxide, lauroyl peroxide, benzoyl peroxide, azobisisobutyronitrile and the like.

Examples of a nonionic surfactant include octylpolyethylene oxide, decylpolyethylene oxide, dodecylpolyethylene oxide, octylpolypropylene oxide, decylpolypropylene oxide, dodecylpolylpropylene oxide and the like.

Examples of a nonionic surfactant containing fluorine include perfluorooctylpolyethylene oxide, perfluorodecylpolyehtylene oxide, perfluorododecylpolyethylene oxide and the like.

Examples of a silane coupling agent include vinyltrimethoxysilane, allyltrimethoxysilane, vinyltriethoxysilane, allyltriethoxysilane, divinyldiethoxysilane, trivinylethoxysilane and the like.

An insulating film can be formed by coating the coating solution on a substrate by a method such as a spin coating method, a roller coating method, a dip coating method, a scan method and the like, and removing an organic solvent by heating treatment.

A method of heating treatment is not particularly limited, but hot plate heating, a method using a furnace, light irradiation heating using a xenon lamp by RTP (Rapid Thermal Processor) and the like can be applied.

A heat treating temperature is preferably, about 200 to about 450° C., more preferably about 250 to about 400° C., and a heating time is usually about 1 minute to about 10 hours.

An insulating film excellent in the mechanical strength and the heat resistance may be formed by further heat treatment to couple Xs in a compound represented by the formula (1) or in a resin obtained by polymerizing the compound in the insulating film, to form a three-dimensional structure.

Alternatively, a porous film may be formed by adding in advance a foaming agent to the coating solution of the present invention.

Examples of a foaming agent to be added include an organic compound having a higher boiling point than that of a solvent of the coating solution, and a resin having a lower thermal degrading temperature than that of a resin obtained by polymerizing the compound represented by the formula (1).

A specific dielectric constant of the thus obtained insulating film is usually 2.5 or lower, being particularly useful for a high speed operating device.

The another embodiment of the present invention is an adamantane resin which is obtained by polymerizing a mixture comprising at least one kind adamantane compound represented by the formula (1), at least one kind adamantane compound represented by the formula (9) and/or at least one kind adamantane compound represented by the formula (10).

In the formulas (9) and (10), B³ and B⁴ represent independently a direct bond, an alkylene group having a carbon number of 1 to 6, an alkenylene group having a carbon number of 2 to 6, or an alkynylene group having a carbon number of 2 to 6. A direct bond is preferable.

Examples of an alkylene group having a carbon number of 1 to 6 include similar groups to B as described above.

Examples of an alkenylene group having a carbon number of 2 to 6 include similar groups to B as described above.

Examples of an alkenylene group having a carbon number of 2 to 6 include similar groups to B as described above.

An alkylene group, an alkenylene group, and an alkynylene group may be straight or branched and, when those groups have a double bond or a triple bond, positions of those bonds are not particularly limited.

Ar⁸ and Ar⁹ represent independently an optionally substituted three or more valent aromatic ring. Examples of a three or more valent aromatic ring include those shown below.

The following formulas show a bond of a naphthalene ring and an anthracene ring may take an arbitrary position.

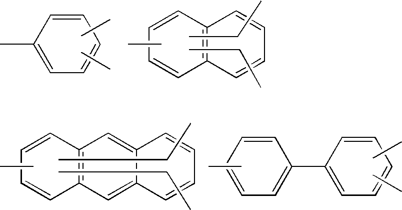

The above aromatic ring may be substituted with at least one kind substituent selected from the group of an alkyl group such as a methyl group, an ethyl group and the like; an alkoxy group such as a methoxy group, an ethoxy group and the like; a halogeno group such as a fluoro group, a chloro group, a bromo group and the like; an acyl group such as an acetyl group, a propionyl group and the like; an alkoxycarbonyl group such as a methoxycarbonyl group, an ethoxycarbonyl group and the like; a hydroxy group; a phenoxy group; a nitro group; a cyano group; a carboxy group; an amino group.

Ar¹⁰ represents an optionally substituted aryl group.

Examples of an aryl group include similar groups to Ar⁴as described above, and these groups may be also substituted with at least one kind substituent described as above.

Y⁸ represents an alkenyl group having a carbon number of 2 to 6 or an alkynyl group having a carbon umber of 2 to 6.

Examples of an alkenyl group having a carbon number of 2 to 6, and alkynyl group having a carbon number of 2 to 6 include similar groups to Y′ as described above.

An alkenyl group and an alkynyl group may be straight or branched, and positions of a double bond and a triple bond are not particularly limited.

Y⁹ represents an alkenylene group having a carbon number of 2 to 6 or an alkynylene group having a carbon number of 2 to 6. Examples of an alkenylene group having a carbon number of 2 to 6 and an alkynylene group having a carbon number of 2 to 6 include those described above.

From the viewpoint of industrial easy availability of starting raw materials for the adamantane compound and easy preparation, it is preferable that B, B³or B⁴is a direct bond.

It is preferable that adamantane compounds represented by the formula (1), (9) and (10) contain at least one carbon-carbon triple bond from the viewpoint that the reactivity at polymerization is enhanced, and it is more preferable that Y¹ and Y⁸ are an alkynyl group having a carbon number of 2 to 6, and Y² and Y⁹ are an alkynylene group having a carbon number of 2 to 6.

In addition, from the viewpoint that adamantane compounds represented by the formulas (1), (9) and (10) form an aromatic ring, a polyvinylene skeleton or a polyacetylene skeleton by polymerization with each other, and the mechanical strength of the resulting insulating film is enhanced, it more preferable that Y¹ and Y⁸ are an ethynyl group, and Y² and Y⁹ are an ethynylene group.

Further, it is particularly preferable that adamantane compounds represented by the formulas (1), (9) and (10) are compounds represented by the formulas (7), (11) and (12) respectively, from the viewpoint that an insulating film having the polarizing property and a low specific dielectric constant may be obtained.

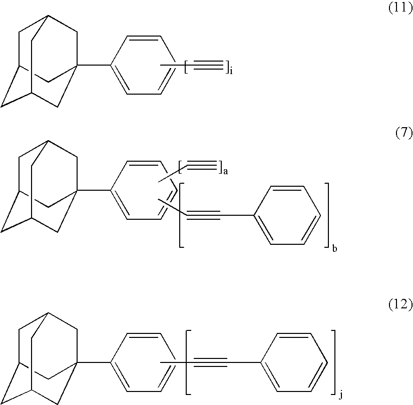

In the formula, a, b, c and d are as defined above.

In adamantane compounds represented by the formulas (1), (9) and (10), it is particularly preferable that i and j are 2 or 3, and it is particularly preferable that a+b of the formula(1) is 2 or 3, from the view point that acetylene, ethynylbenzene, diethynyl benzene and the like which are raw materials may be easily obtained.

Examples of a compound represented by the formula (9) include diethynylphenyladamantane, triethynylphenyladamantane, tetraethynylphenyladamantane, pentaethynylphenyladamantane, and the like.

Examples of a compound represented by the formula (10) include bis(phenylethynyl)phenyladamantane, tris(phenylethynyl)phenyladamantane, tetrakis(phenylethynyl)phenyladamantane, pentakis(phenylethynyl)phenyladamantane and the like.

Among adamantane compounds represented by the formulas (9) and (10), compounds having a substituent at a methylene group of adamantane may be prepared, for example, by the same method as described above

In addition, among adamantane compounds represented by the formulas (9) and (10), compounds having a substituent at a bridging methine group of adamantane can be prepared, for example, by the same method as described above.

A coating solution can be obtained by dissolving an adamantane resin obtained by polymerizing a mixture comprising at least one kind adamantane compound represented by the formula (1), at least one kind adamantane compound represented by the formula (9) and/or at least one kind adamantane compound represented by the formula (10).

Alternatively, a coating solution of the present invention may be obtained by polymerizing in a solution obtained by dissolving a mixture comprising at least one kind adamantane compound represented by the formula (1), at least one kind adamantane compound represented by the formula (9) and/or at least one kind adamantane compound represented by the formula (10) in an organic solvent.

A polymerization method includes the same method described above.

Examples of an organic solvent used for obtaining a coating solution include the same as described above.

Further, similar additives as described above may be added to the coating solution in a range that there activity, the coating property and the like of compounds represented by the formulas (1), (9) and (10) are not deteriorated.

An insulating film can be formed by coating the coating solution on a substrate by an arbitrary method such as a spin coating method, a roller coating method, a dip coating method, a scan method and the like, and removing an organic solvent by heating treatment.

A method of heating treatment is not particularly limited, but hot plate heating, a method using a furnace, light irradiation heating using a xenon lamp by RTP (Rapid Thermal Processor) and the like can be applied.

A heat treating temperature is preferably about 200 to about 450° C., more preferably about 250 to about 400° C., and a heating time is usually about 1 minute to about 10 hours.

An insulating film excellent in the mechanical strength and the heat resistance may be formed by further heat treatment, whereby, at least two selected from the group consisting of Y¹, Y², Y⁸ and Y⁹ in a resin are coupled with each other to form a three-dimensional structure.

Alternatively, a porous film may be formed by adding in advance a foaming agent to the coating solution.

Examples of a foaming agent include the same as described above.

A specific dielectric constant of the thus obtained insulating film is usually 2.5 or lower, being particularly useful for a high speed operating device.

The other embodiment of the present invention is an coating solution comprises a resin obtained by polymerizing at least one kind adamantane compound represented by the above formula (1) and at least one adamantane compound selected from the group consisting of adamantane compound represented by the formula (1) and adamantane compound represented by the formula(6).

In the formulas (6), B² represents a direct bond, an alkylene group having a carbon number of 1 to 6, an alkenylene group having a carbon number of 2 to 6, or an alkynylene group having a carbon number of 2 to 6. B² is preferably a direct bond.

Examples of an alkylene group having a carbon number of 1 to 6, an alkenylene group having a carbon number of 2 to 6, and an alkynylene group having a carbon number of 2 to 6 include the similar groups to B as described above.

An alkylene group, an alkenylene group, and an alkynylene group may be straight or branched and, when those groups have a double bond or a triple bond, positions of those bonds are not particularly limited.

Y⁷ represents an alkenyl group having a carbon number of 2 to 6 or an alkynyl group having a carbon number of 2 to 6.

Examples of an alkenyl group having a carbon number of 2 to 6 and an alkynyl group having a carbon number of 2 to 6 include the similar groups to Y¹ as described above.

From the viewpoint of industrial easy availability of starting raw materials for the adamantane compound and easy preparation, it is preferable that B and B² are direct bond.

When B² is direct bond, a substituent-Ar⁷—[Y⁷]ₕ is preferably at a methine position of adamantane.

It is preferable that adamantane compounds represented by the formula (6) contain at least one carbon-carbon triple bond from the viewpoint that the reactivity at polymerization is enhanced, and it is more preferable that Y⁷ is an alkynyl group having a carbon number of 2 to 6.

In addition, from the viewpoint that adamantane compounds represented by the formulas (6) form an aromatic ring, a polyvinylene skeleton or a polyacetylene skeleton by polymerization with each other, and that the mechanical strength of the resulting insulating film is enhanced, it is more preferable that Y¹ and Y³ are an ethynyl group, and Y² is an ethynylene group.

Further, it is particularly preferable that adamantane compounds represented by the formula (1) and (6) are compounds represented by the formulas (7) and (8) respectively, because an insulating film having the polarizing property and a low specific dielectric constant is obtained.

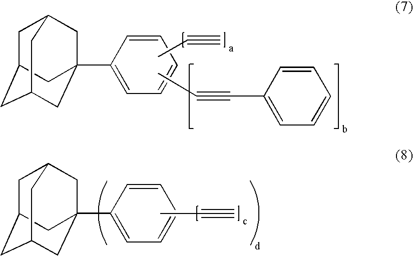

In the formula, a, b, c and d are as defined above.

In adamantane compounds represented by the formulas (1) and (6), it is particularly preferable that a+b is 2 or 3, g is an integer of 1 to 3, and h is 2 or 3, from the viewpoint that acetylene, ethynylbenzene, diethynyl benzene and the like which are raw materials may be easily obtained.

Examples of the adamantane compound represented by the formula (6) include bis(ethynylphenyl)adamantane,

tris(ethynylphenyl)adamantane,

tetrakis(ehynylphenyl)adamantane,

bis(diethynylphenyl)adamantane,

tris(diethynylphenyl)adamantane,

tetrakis(diethynylphenyl)adamantane,

bis(triethynylphenyl)adamantane,

tris(triehtnylphenyl)adamantane,

tetrakis(triethynylphenyl)adamantane,

bis(tetraethynylphenyl)adamantane,

tris(tetraethynylphenyl)adamantane,

tetrakis(tetraethynylphenyl)adamantane, and the like.

The compound represented by the formula (6) can be prepared, for example, by reacting the same method as described above.

A coating solution can be obtained by dissolving in a organic solvent a resin obtained by polymerizing at least one kind of adamantane compounds represented by the formula (1), and at least one adamantane compound selected from the group consisting of, adamantane compounds represented by the formula (1) and adamantane compounds represent by the formula (6).

The coating solution of the present invention may be obtained by partially polymerizing a solution obtained by dissolving at least one kind adamantane compound represented by the formula (1) in an organic solvent, that is, by polymerization so that an adamantane compound monomer represented by the formula (1) remains and, as a result, an adamantane resin and an adamantane monomer coexist, and optionally further adding at least one kind adamantane compound represented by the formula (6) to this.

The coating solution may be obtained by dissolving in an organic solvent a resin obtained by polymerizing a mixture comprising at least one adamantane compound represented by the formula (1), and at least one adamantane compound represented by the formula (6).

The coating solution of the present invention may be obtained by polymerizing in a solution obtained by dissolving at least one kind adamantane compound represented by the formula (1), and optionally at least one kind adamantane compound represented by the formula (6) in an organic solvent.

A polymerization method includes the same method as described above.

An insulating film can be formed by the same method as described above.

An insulating film excellent in the mechanical strength and the heat resistance can be formed by heat treatment, whereby, at least two selected from the group consisting of Y¹, Y² and Y⁷ in a resin are coupled to form a three-dimensional structure.

A heat treating temperature is preferably about 200 to about 450° C., more preferably about 250 to about 400° C., and a heating time is usually about 1 minute to about 10 hours.

Alternatively, a porous film may be formed by in advance adding a foaming agent to the coating solution of the present invention.

Examples of a forming agent include the same as described above.

A specific dielectric constant of the thus obtained insulating film is usually 2.5 or lower, being particularly useful for a high speed operating device.

### EXAMPLES

The present invention will be explained in more detail byway of Examples below, and the present invention is not limited by Examples.

### Preparation 1

20.0 g of 1-bromoadamantane, 4.6 g of aluminium bromide and 100 mL of 1,3-dibromobenzene were placed into a 300 mL of four-necked flask, and the mixture was stirred overnight while maintaining at 0° C. with an ice bath. Thereafter, 100 mL of 1N hydrochloric acid was placed, the mixture was stirred well, and the organic layer was taken out, and washed with 100 mL of water. About 400 mL of methanol was added to the organic layer, the precipitated crystals were filtered, and dried to obtain 22.4 g of 1-(3,5-dibromophenyl)adamantane.

### Preparation 2

10.0 g of 1-(3,5-dibromophenyl)adamantane obtained in Preparation 1, 220 mg of dichlorobis(triphenylphosphine)palladium, 420 mg of triphenylphosphine, 180 mg of copper (I) iodide and 100 mL of triethylamine were placed into a 200 mL four-necked flask, and 6.4 g of trimethylsilylacetylene was added dropwise over 1 hour while maintaining at 85° C. Thereafter, stirring continued at the same temperature for 4 hours. After allowing cooling, the solvent was distilled off under reduced pressure, 150 mL of diethyl ether was added to the residue, and the insolubles were filtered. The filtrate was successively washed with 100 mL of 1N hydrochloric acid, 100 mL×2 of water and 100 mL of a saturated brine, and dried with anhydrous sodium sulfate. A desiccant was filtered, and the solvent was removed to obtain the crude product. The crude product was purified by column chromatography to obtain 8.8 g of 1-(3,5-di(trimethylsilylethynyl)phenyl)adamantane.

### Preparation 3

8.8 g of 1-(3,5-di(trimethylsilylethynyl)phenyl)adamantane obtained in Preparation 2 was placed into a 500 mL flask, 200 mL of methanol, 100 mL of THF and 0.5 g of anhydrous potassium carbonate were placed, and the mixture was stirred at room temperature for about 2 hours. The solvent was completely removed by an evaporator, the residue was dissolved in 200 mL of methylene chloride, and washed with 100 mL of 1N hydrochloric acid and 100 mL of water. The organic layer was dried with anhydrous magnesium sulfate, an desiccant was filtered, and the solvent was removed to obtain 5.5 g of 1-(3,5-diethynylphenyl)adamantane.

### Preparation 4

2.0 g of 1-(3,5-diethynylphenyl)adamantane obtained in Preparation 3 and 48 g of anisole were placed into a 100 mL flask, and the mixture was heated for 18 hours while refluxing, to obtain poly(1-(3,5-diethynylphenyl)adamantane). As the result of GPC analysis, a weight average molecular weight in terms of polystyrene of this resin was about 5000.

### Example 1

1-(3,5-diethynylphenyl)adamantane obtained in Preparation 3 was dissolved in anisole, to solid matters of 10%. Then, the material was filtered with a 0.2 μm filter to prepare a coating solution. The resulting coating solution was spin-coated on a 4-inch silicon wafer at a rotation number of 2000 rpm, pre-baked at 150° C. for 1 minute, and heat-treated at 350° C. for 30 minutes. A specific dielectric constant of the resulting insulating film was measured with a mercury probe method (Nihon SSM, SSM495) and found to be 2.46.

### Example 2

Poly(1-(3,5-diethynylphenyl)adamantane) obtained in Preparation 4 was dissolved in anisole, to solid matters of 10%. Then, the material was filtered with a 0.2 μm filter to prepare a coating solution. The resulting coating solution was spin-coated on a 4-inch silicon wafer at a rotation number of 2000 rpm, pre-baked at 150° C. for 1 minute, and heat-treated at 350° C. for 30 minutes. A specific dielectric constant of the resulting insulating film was measured with a mercury probe method (Nihon SSM, SSM495) and found to be 2.38.

### Preparation 5

10.0 g of 1-(3,5-dibromophenyl)adamantane obtained in Preparation 1,220 mg of dichlorobis(triphenylphosphine)palladium, 420 mg of triphenylphosphine, 180 mg of copper (I) iodide and 100 mL of triethylamine were placed into a 200 mL four-necked flask, and a mixture of 3.2 g of trimethylsilylacetylene and 3.3 g of ethynylbenzene was added dropwise over 1 hour while maintaining at 85° C. Thereafter, stirring continued at the same temperature for 4 hours. After allowing to cool, the solvent was distilled off under reduced pressure, 150 mL of diethyl ether was added to the residue, and the insolubles were filtered. The filtrate was successively washed with 100 mL of 1N hydrochloric acid, 100 mL×2 of water and 100 mL of a saturated brine, and dried with anhydrous sodium sulfate. A desiccant was filtered, and the solvent was removed to obtain the crude product. The crude product was purified by column chromatography to obtain 9.6 g of a mixture of 1-(3,5-di(trimethylsilylethynyl)phenyl)adamantane, 1-(3-(trimethylsilylethynyl)-5-(phenylethynyl)phenyl)adamantane and 1-(3,5-di(phenylethynyl)phenyl)adamantane.

### Preparation 6

9.6 g of the mixture obtained in Preparation 5 was placed into a 500 mL flask, 200 mL of methanol, 100 mL of THF and 0.5 g of anhydrous potassium carbonate were placed, and the mixture was stirred at room temperature for about 2 hours. The solvent was completely removed by an evaporator, the residue was dissolved in 200 mL of methylene chloride, and washed with 100 mL of 1N hydrochloric acid and 100 mL of water. The organic layer was dried with anhydrous magnesium sulfate, an desiccant was filtered, and the solvent was removed to obtain 7.6 g of a mixture of 1-(3,5-diethynylphenyl)adamantane, 1-(3-ethynyl-5-(phenylethynyl)phenyl)adamantane and 1-(3,5-di(phenylethynyl)phenyl)adamantane. This is designated as monomer A.

### Preparation 7

2.0 g of the mixture obtained in Preparation 6 and 18 g of anisole were placed into a 100 mL flask, and the mixture was heated for 100 hours while refluxing, to obtain an adamantane resin solution. As the result of GPC analysis, a weight average molecular weight in terms of polystyrene of this resin was about 2500.

### Preparation 8

85.5 g of an adamantane resin solution obtained as in Preparation 7 was charged into 1 kg of methanol, and the mixture was stirred for 1 hour. The precipitated powders were filtered off, and dried under reduced pressure to obtain 4.5 g of an adamantane resin. As the result of GPC analysis, a weight average molecular weight in terms of polystyrene of this resin was about 2800.

### Example 3

Anisole was added to the adamantane resin solution obtained in Preparation 7, to solid matters of 10%. Then, the material was filtered with a 0.2 μm filter to prepare a coating solution. The resulting coating solution was spin-coated on a 4 inch silicon wafer at a rotation number of 2000 rpm, pre-baked at 150° C. for 1 minutes, and heat-treated at 400° C. for 30 minutes. A specific dielectric constant of the resulting insulating film was measured with a mercury probe method (Nihon SSM, SSM495) and found to be 2.34.

### Example 4

The adamantane resin obtained in Preparation 8 was dissolved in anisole to solid matters of 10%. Then, the material was filtered with a 0.2 μm filter to prepare a coating solution. The resulting coating solution was spin-coated on a 4 inch silicon wafer at a rotation number of 2000 rpm, pre-baked at 150° C. for 1 minute, and heat-treated at 400° C. for 30 minutes to obtain an insulating film. The heat resistance of this insulating film was assessed by a thermal cycle test of 400° C.×30 minutes, and a decrease rate of a film per unit cycle was 0.3% or smaller, being excellent in the heat resistance.

### Preparation 9

According to the same manner as that of Preparation 5 except that 5.0 g of 1-(3,5-dibromophenyl)adamantane, 80 mg of dichlorobis(triphenylphosphine)palladium, 170 mg of triphenylphosphine, 60 mg of copper (I) iodide, 100 mL of triethylamine, 1.2 g of trimethylsilylacetylene and 1.8 g of ethynylbenzene were used, 3.4 g of a mixture of 1-(3,5-di(trimethylsilylethynyl)phenyl)adamantane, 1-(3-(trimethylsilylethynyl)-5-(phenylethynyl)phenyl)adamantane and 1-(3,5-di(phenylethynyl)phenyl)adamantane was obtained.

### Preparation 10

According to the same manner as that of Preparation 6 except that 3.4 g of the mixture obtained in Preparation 3, 30 ml of methanol, 15 mL of THF and 0.1 g of anhydrous potassium carbonate were used, 2.6 g of a mixture of 1-(3,5-diethynylphenyl)adamantane, 1-(3-ethynyl-5-(phenylethynyl)phenyl)adamantane and 1-(3,5-di(phenylethynyl)phenyl)adamantane was obtained. This is designated as monomer B.

### Preparation 11

4.0 g of the monomer A and 36 g of N-methylpyrrolidone were placed into a 100 mL flask, and the mixture was stirred for 50 hours while maintaining at 200° C. 20 g of the resulting solution was charged into 200 ml of methanol, followed by stirring for 1 hour. The precipitated powders were filtered to take out, and dried under reduced pressure to obtain 1.3 g of an adamantane resin. As result of GPC analysis, a weight average molecular weight of this resin in terms of polystyrene was about 3000.

### Preparation 12

2.0 g of the monomer B and 18 g of anisole were placed into a 100 mL flask, and the mixture was heated for 100 hours while refluxing, to obtain an adamantane resin solution. As the result of GPC analysis, this solution contained 33% of a resin having a weight average molecular weight in terms of polystyrene of about 2500, and 67% of a raw material monomer B.

### Example 5

0.5 g of the adamantane resin obtained in Preparation 11, and 1.0 g of the monomer A were dissolved in anisole, to solid matters of 10%. Then, the material was filtered with a 0.2 μm filter to prepare a coating solution. The resulting coating solution was spin coated on a 4-inch silicon wafer at a rotation number of 2000 rpm, pre-baked at 150° C. for 1 minute, and heat-treated at 400° C. for 30 minutes to obtain an insulating film. The heat resistance of this insulating film was assessed by a thermal cycle test of 40° C.×30 minutes, and a decrease rate of a film per unit cycle was 0.2% or smaller, being excellent in the heat resistance.

### Example 6

Anisole was added to the adamantane resin solution obtained in Preparation 12, to solid matters of 10%. Then, the material was filtered with a 0.2 μm filter to prepare a coating solution. The resulting coating solution was spin-coated on a 4-inch silicon wafer at a rotation number of 2000 rpm, pre-baked at 150° C. for 1 minute, and heat-treated at 400° C. for 30 minutes. A specific dielectric constant of the resulting insulating film was measured by a mercury probe method (Nihon SSM, SSM495), and was found to be 2.27.

### Preparation 13

200 g of bromine and 0.43 g of electrolytic iron were placed into a 1L four-necked flask, and 34.1 of adamantane was gradually placed therein while ice-cooling. Thereafter, the material was diluted with 400 mL of methylene chloride, a 10% aqueous sodium sulfite solution was added, and the mixture was stirred well. The aqueous layer was removed, and the organic layer was washed with 200 mL of water two times. The organic layer was concentrated, 50 mL of diethyl ether was added to the resulting crude product, 500 mL of methanol was further added, and the mixture was stirred well. The precipitated crystals were filtered, and dried to obtain 46.2 g of 1,3-dibromoadamantane.

### Preparation 14

5.0 g of 1,3-dibromoadamantane, 2.3 g of aluminium bromide and 100 mL of 1,3-dibromobenzene were placed into a 300 mL four-necked flask, and the mixture was stirred overnight while maintaining at 0° C. with an ice bath. Thereafter, a temperature was retained at 60° C. for 4 hours. After cooling, 100 mL of 1N hydrochloric acid was placed, the mixture was stirred well, and the organic layer was taken out, and washed with 100 mL of water. About 400 mL of methanol was added to the organic layer, the precipitated crystals were filtered and dried to obtain 6.8 g of 1,3-bis(3,5-dibromophenyl)adamantane.

### Preparation 15

6.0 g of 1,3-bis (3,5-dibromophenyl)adamantane obtained in Preparation 14, 200 mg of dichlorobis(triphenylphosphine)palladium, 400 mg of triphenylphosphine, 180 mg of copper (I) iodide and 100 mL of triethylamine were placed into a 200 mL four-necked flask, and 6.5 g of trimethylsilylacetylene was added dropwise over 1 hour while maintaining at 85° C. Thereafter, stirring continued at the same temperature for 4 hours. After allowing to cool, the solvent was removed under reduced pressure, 150 mL of diethyl ether was added to the residue, and the insolubles were filtered. The filtrate was successively washed with 100 mL of 1N hydrochloric acid, 100 mL×2 of water and 100 mL of a saturated brine, and dried with anhydrous sodium sulfate. A desiccant was filtered, and the solvent was removed to obtain 5.9 g of the crude product. The crude product was purified by column chromatography to obtain 1,3-bis(3,5-di(trymethylsilylethynyl)phenyl)adamantane.

### Preparation 16

According to the same manner as that of Preparation 15 except that 10.0 g of 1-(3,5-dibromophenyl)adamantane obtained in Preparation 3, 220 mg of dichlorobis(triphenylphosphine)palladium, 420 mg of triphenylphosphine, 180 mg of copper (I) iodide, 100 mL of triethylamine, and 6.4 g of trymethylsilylacetylene were used, 8.8 g of 1-(3,5-di (trimethylsilylethynyl)phenyl)adamantane was obtained.

### Preparation 17

5.9 g of 1,3-bis(3,5-di(trimethylsilylethynyl)phenyl)adamantane obtained in Preparation 15 was placed into a 500 mL flask, 150 mL of methanol, 100 mL of THF, and 0.5 g of anhydrous potassium carbonate were placed, and the mixture was stirred at room temperature for about 2 hours. The solvent was completely removed with an evaporator, the residue was dissolved in 200 mL of methylene chloride, and washed with 100 mL of 1N hydrochloric acid and 100 mL of water. The organic layer was dried with anhydrous magnesium sulfate, a desiccant was filtered, and the solvent was removed to obtain 3.2 g of 1,3-bis(3,5-diethynylphenyl)adamantane.

### Preparation 18

According to the same manner as that of Preparation 5 except that 8.8 g of 1-(3,5-di(trimethylsilylethynyl)phenyl)adamantane obtained in Preparation 5, 200 mL of methanol, 100 mL of THF and 0.5 g of anhydrous potassium carbonate were used, 5.5 g of 1-(3,5-diethylphenyl)adamantane was obtained.

### Preparation 19

According to the same manner as that of Preparation 5 except that 9.6 g of the mixture obtained in Preparation 6, 200 mL of methanol, 100 mL of THF and 0.5 g of anhydrous potassium carbonate were used, 7.6 g of a mixture of 1-(3,5-diethynylphenyl)adamantane, 1-(3-ethynyl-5-(phenylethynyl)phenyl)adamantane and 1-(3,5-di(phenylethynyl)phenyl)adamantane was obtained.

### Preparation 20

21 g of 1-(3,5-diethynylphenyl)adamantane obtained as in Preparation 18 and 49 g of anisole were placed into a 200 mL flask, and the mixture was heated for 5 hours while refluxing, to obtain an adamantane resin solution. As the result of GPC analysis, a weight average molecular weight in terms of polystyrene of this resin was about 6800.

### Preparation 21

2.0 g of the mixture obtained in Preparation 19 and 18 g of anisole was placed into a 100 mL of flask, and the mixture was heated for 100 hours while refluxing, to obtain an adamantane resin solution. As the result of GPC analysis, a weight average molecular weight in terms of polystyrene of this resin was about 2500.

### Example 7

1,3-bis(3,5-diethynylphenyl)adamantane obtained in Preparation 17 was added to the adamantane resin solution obtained in Preparation 20, and anisole was further added to solid matters of 15%. Then, the material was filtered with a 0.2 μm filter to prepare a coating solution. The resulting coating solution was spin-coated on a 4-inch silicon wafer at a rotation number of 2000 rpm, pre-baked at 150° C. for 1 minute, and heat-treated at 400° C. for 30 minutes to obtain an insulating film. The heat resistance of this insulating film was assessed by a thermal cycle test of 400° C.×30 minutes, a decrease rate of a film per unit cycle was less than 0.1%, and substantial film decrease was not observed, being very excellent in the heat resistance.

### Example 8

1,3-bis(3,5-diethynylphenyl)adamantane obtained in Preparation 17 was added to the adamantane resin solution obtained in Preparation 21, and anisole was further added to solid matters of 10%. Then, the material was filtered with a 0.2 μm filter to prepare a coating solution. The resulting coating solution was spin-coated on a 4-inch silicon wafer at a rotation number of 2000 rpm, pre-baked at 150° C. for 1 minute, and heat-treated at 400° C. for 30 minutes. A specific dielectric constant of the resulting insulating film was observed by a mercury prove method (Nihon SSM, SSM495), and was found to be 2.37.

According to the present invention, there can be provided a novel compound which can prepare an insulating film having a low dielectric constant.

Further according to the present invention, there can be prepared an insulating film forming coating solution which can prepare an insulating film which has a low dielectric constant and excellent in the heat resistance.

What is claimed is:

1. An adamantane compound represented by the formula (1). 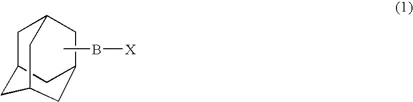
(wherein B represents a direct bond, an alkylene group having a carbon number of 1 to 6, an alkenylene group having a carbon number of 2 to 6, or an alkynylene group having a carbon number of 2 to 6, and X represents an organic group represented by the formula (2). 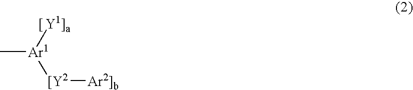
(wherein Ar¹ represents an optionally substituted three or more valent aromatic ring, Ar² represents an optionally substituted aryl group or an organic group represented by the formula (3), Y¹ represents an alkenyl group having a carbon number of 2 to 6or an alkynyl group having a carbon number of 2 to 6, Y² represents an alkenylene group having a carbon number of 2 to 6 or an alkynylene group having a carbon number of 2 to 6, and a and b represent an integer of 0 to 5, respectively, a+b is from 2 to 5.) 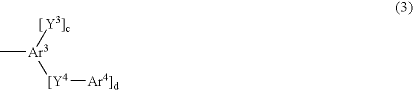
(wherein Ar³ represents an optionally substituted two or more valent aromatic ring, Ar⁴represents an optionally substituted aryl group or an organic group represented by the formula (4), Y³ represents an alkenyl group having a carbon number of 2 to 6or an alkynyl group having a carbon number of 2to 6, Y⁴ represents an alkenylene group having a carbon number of 2 to 6 or an alkynylene group having a carbon number of 2 to 6, and c and d represent an integer of 0 to 5, respectively, c+d is from 1 to 5.) 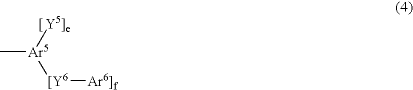
(wherein Ar⁵ represents an optionally substituted two or more valent aromatic ring, Ar⁶represents an optionally substituted aryl group, Y⁵ represents an alkenyl group having a carbon number of 2 to 6 or an alkynyl group having a carbon number of 2 to 6, Y⁶ represents an alkenylene group having a carbon number of 2 to 6 or an alkynylene group having a carbon number of 2 to 6, and e and f represent an integer of 0 to 5, respectively, e+f is from 1 to 5.)

2. The adamantane compound according to claim 1, wherein the compound represented by the formula (1) is a compound represented by the formula (5). 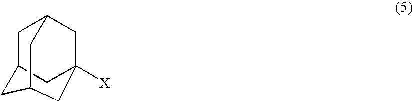
(wherein X is as defined above).

3. The adamantane compound according to claim 1, wherein Y¹ is an alkynyl group having a carbon number of 2 to 6, and Y² is an alkynylene group having a carbon number of 2 to 6.

4. The adamantane compound according to claim 1, wherein X is an organic group selected from the following group. 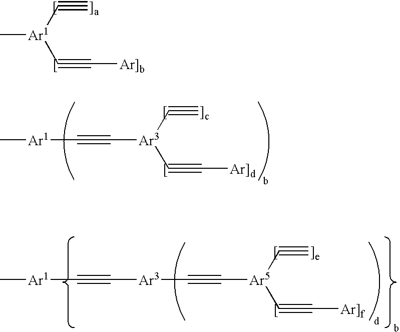
(wherein Ar¹, Ar³, Ar⁵, a, b, c, d, e and f are as defined above, and Ar represents an optionally substituted aryl group.)

5. The adamantane compound according to claim 4, wherein X is an organic group selected from the following group. 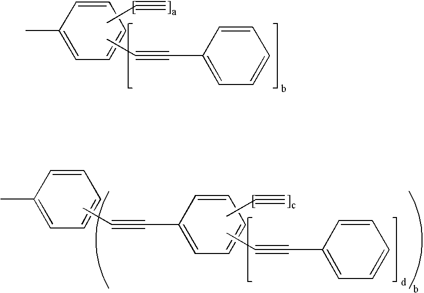
(wherein a, b, c and d are as defined above.)

6. The adamantane compound according to claim 1, wherein X is an organic group selected from the following groups.

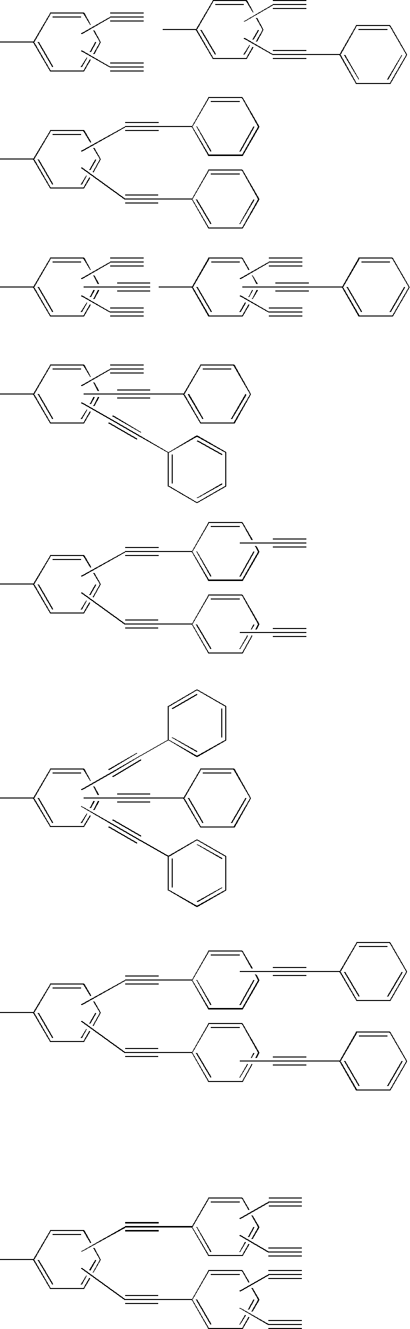

7. A resin obtained by polymerizing the adamantane compound according to claim 1.

8. A coating solution comprising the resin according to claim 7 and at least one adamantane compound selected from the group consisting of the adamantane compound represented by the above formula (1) and at least one kind adamantane compound represented by the formula (6). 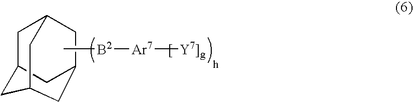
(wherein B² represent a direct bond, an alkylene group having a carbon number of 1 to 6, an alkenylene group having a carbon number of 2 to 6 or an alkynylene group having a carbon number of 2 to 6, Ar⁷ represents an optionally substituted two or more valent aromatic ring, Y⁷ represent an alkenyl group having a carbon number of 2 to 6 or an alkynyl group having a carbon number of 2 to 6, g represents an integer of 1 to 5, and h represents an integer of 2 to 4).

9. A method for forming an insulating film comprising coating the coating solution according to claim 8 and heat treatment.

10. The method according to claim 9, wherein three-dimensional cross-linking is performed after heat treatment.

11. The coating solution according to claim 8, wherein the compound represented by the formula (1) is a compound represented by the formula (5). 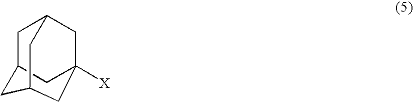
(wherein X is as defined above).

12. The coating solution according to claim 8, wherein Y¹ of the compound represented by the formula (1) is an alkynyl group having a carbon number of 2 to 6, and Y² of the compound represented by the formula (1) is an alkynylene group having a carbon number of 2 to 6.

13. The coating solution according to claim 12, wherein Y¹ is an ethynyl group, and Y² is an ethynylene group.

14. The coating solution according to claim 8, wherein the compound represented by the formula (1) is a compound represented by the formula (7). 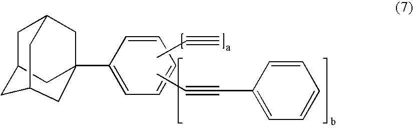
(wherein a and b are as defined above)

15. The coating solution according to claim 8, wherein a+b of the above formula (1) is 2 or 3.

16. A coating solution comprising a resin obtained by polymerizing a mixture comprising at least one adamantane compound represented by the above formula (1), and at least one adamantane compound represented by the formula (6).

17. The coating solution according to claim 8 or 16, wherein B² is a direct bond.

18. The coating solution according to claim 16, wherein B is a direct bond.

19. The coating solution according to claim 16, wherein Y¹ of the compound represented by the formula (1) is an alkynyl group having a carbon number of 2 to 6, and Y² of the compound represented by the formula (1) is an alkynylene group having a carbon number of 2 to 6.

20. The coating solution according to claim 16, wherein Y¹ is an ethynyl group, and Y ² is an ethynylene group.

21. The coating solution according to claim 16, wherein the adamantane compound represented by the formula (1) is a compound represented by the formula (7), and the adamantane compound represented by the formula (6) is a compound represented by the formula (8). (wherein a, b, c and d are as defined above.)

22. The coating solution according to claim 8 or 16, wherein a+b is 2 or 3, c is an integer of 1 to 3, and d is 2 or 3.

23. A method for forming an insulating film comprising coating the coating solution according to claims 8 or 16 on a substrate, and heat treatment.

24. The method according to claim 23, wherein three-dimensional cross-linking is performed after heat treatment.

25. An adamantane resin, which is obtained by polymerizing a mixture comprising at least one adamantane compound represented by the formula (1), at least one adamantane compound represented by the formula (9) and/or at least one adamantane compound represented by the formula (10). 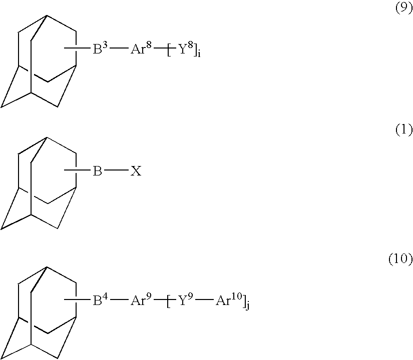
(wherein B and X are as defined above, B³ and B⁴ represent independently a direct bond, an alkylene group having a carbon number of 1 to 6, an alkenylene group having a carbon number of 2 to 6 or an alkynylene group having a carbon number of 2 to 6, Ar⁸ and Ar⁹ represent independently an optionally substituted three or more valent aromatic ring, Ar¹⁰ represents an optionally substituted aryl group, Y⁸ represents an alkenyl group having a carbon atom of 2 to 6 or an alkynyl group having a carbon number of 2 to 6, Y⁹ represents an alkenylene group having a carbon number of 2 to 6 or an alkynylene group having a carbon number of 2 to 6, and i and j represent an integer of 2 to 5.)

26. The adamantane resin according claim 25, wherein B, B³ and B⁴ are a direct bond.

27. The adamantane resin according to claim 25, wherein Y¹ and Y⁸ are an alkynyl group having a carbon number of 2 to 6, and Y² and Y⁹ are an alkynylene group having a carbon number of 2 to 6.

28. The adamantane resin according to claim 27, wherein Y¹ and Y⁸ are an ethynyl group, and Y³ and Y⁹ are an ethynylene group.

29. The atamantane resin according to claim 25, wherein the adamantane compound represented by the formula (a) is a compound represented by the formula (11), the adamantane compound represented by the formula (1) is a compound represented by the formula (7), and the adamantane compound represented by the formula (10) is a compound represented by the formula (12). (wherein a, b, i and j are as defined above.)

30. The adamantane resin according to claim 25, wherein i and j are 2 or 3, respectively, and a+b of the formula (1) is 2 or 3.

31. A coating solution comprising an adamantane resin according to claim 25.

32. A method for forming an insulating film comprising coating the coating solution according to claim 31 and heat treatment.

33. The method according to claim 32, wherein three-dimensional cross-linking is performed after heat treatment.
